(19) United States
(12) Patent Application Publication
(10) Pub. No.: US 2020/0337904 A1
(43) Pub. Date: Oct. 29, 2020

(54) DRESSING INCLUDING DEHYDRATED PLACENTAL TISSUE FOR WOUND HEALING

(71) Applicants: SYSTAGENIX WOUND MANAGEMENT, LIMITED, West Sussex (GB); KCI USA, INC., San Antonio, TX (US)

(72) Inventor: Alexander WAITE, West Sussex (GB)

(21) Appl. No.: 16/955,584

(22) PCT Filed: Dec. 19, 2018
(86) PCT No.: PCT/US18/66573

§ 371 Date: Jun. 18, 2020

Related U.S. Application Data
(60) Provisional application No. 62/608,461, filed on Dec. 20, 2017.

(51) Int. Cl. A61F 13/00; A61M 1/00; A61L 15/40; A61L 15/22; A61L 15/44; A61L 15/42

(52) U.S. Cl. CPC A61F 13/00029; A61F 13/00068; A61F 13/00012; A61M 1/0088; A61F 13/00063; A61L 2300/104; A61L 15/40; A61L 15/225; A61L 15/44; A61L 15/425; A61L 2300/404; A61F 13/00987

(57) ABSTRACT

A dressing for wound healing is provided herein including dehydrated placental tissue, collagen, and oxidized regenerated cellulose. The dehydrated placental tissue may be present in a first layer and the collagen and the oxidized regenerated cellulose may be combined into a second layer. The dehydrated placental tissue may comprise amniotic membrane tissue, chorion tissue, or a combination thereof. The second layer including the collagen and the oxidized regenerated cellulose may comprise about 50% to about 60% collagen by weight and about 40% to about 50% ORC by weight.

### CROSS-REFERENCE TO RELATED APPLICATIONS

This application claims the benefit of and priority to U.S. Provisional Patent Application No. 62/608,461, filed Dec. 20, 2017, the entire contents of which are incorporated herein by reference.

### TECHNICAL FIELD

The claimed subject matter relates generally to treatment of tissue, including without limitation compositions, dressings, and other apparatuses for application to a tissue site, such as a wound.

### BACKGROUND

A wide variety of materials and devices, generally characterized as “dressings,” are generally known in the art for use in treating an injury, defect, or other disruption of tissue. Such disruptions of tissue may be the result of trauma, surgery, or disease, and may affect skin or other tissues. In general, dressings may control bleeding, absorb exudate, ease pain, assist in debriding tissue, protect tissue from infection, or otherwise promote healing and protect tissue from further damage.

Some dressings may protect tissue from, or even assist in the treatment of, infections associated with wounds. Infections can retard wound healing and, if untreated, can result in tissue loss, systemic infections, septic shock, and death. While the benefits of dressings are widely accepted, improvements to dressings may benefit healthcare providers and patients.

### BRIEF SUMMARY

In one aspect, the present disclosure provides a dressing that includes dehydrated placental tissue, a collagen, and an oxidized regenerated cellulose.

In some embodiments, the dehydrated placental tissue may be present in a first layer and the collagen and the ORC may be combined into a second layer.

In some embodiments, the dehydrated placental tissue may include amniotic membrane tissue, chorion tissue, or a combination thereof.

In some embodiments, the second layer comprises about 50% to about 60% collagen by weight. Additionally or alternatively, in some embodiments, the second layer which includes the collagen and the ORC may include about 50%, about 51%, about 52%, about 53%, about 54%, about 55%, about 56%, about 57%, about 58%, about 59%, about 60%, or any range including and/or in between any two of these values, collagen by weight.

In some embodiments, the second layer comprises about 40% to about 50% ORC by weight. Additionally or alternatively, in some embodiments, the second layer which includes the collagen and the ORC may include about 40%, about 41%, about 42%, about 43%, about 44%, about 45%, about 46%, about 47%, about 48%, about 49%, about 50%, or any range including and/or in between any two of these values, ORC by weight.

In some embodiments, the second layer is in a freeze-dried form. In some embodiments, the second layer is in the form of a film. In some embodiments, the second layer is in the form of a sponge.

In some embodiments, the second layer is configured as a wound contact layer.

In some embodiments, the second layer includes an antimicrobial agent. Additionally or alternatively, in some embodiments, the antimicrobial agent may be one or more of tetracycline, penicillins, terramycins, erythromycin, bacitracin, neomycin, polymycin B, mupirocin, clindamycin, colloidal silver, silver sulfadiazine, chlorhexidine, povidone iodine, triclosan, sucralfate, quaternary ammonium salts, pharmaceutically acceptable silver salts, or any combination thereof.

In some embodiments, the second layer may include silver and at least a portion of the silver may be present in a complex with the ORC (silver-ORC).

In some embodiments, the second layer may include an antioxidant. Additionally or alternatively, in some embodiments, the antioxidant may be one or more of anthocyanins, astaxanthin, bilirubin, canthaxanthin, capsaicin, citric acid, curcumin, coenzyme Q10, eugenol, flavanols, flavonolignans, flavanones, flavones, flavonols, iodide, isoflavone phytoestrogens, lutein, lycopene, manganese, melatonin, N-acetylcysteine, oxalic acid, phenolic acids, phytic acid, R-α-lipoic acid, stilbenoids, tocopherol, tocotrienol, vitamin A, vitamin C, vitamin E, xanthones, zeaxanthin, α-carotene, β-carotene, or any combination thereof.

In some embodiments, the dressing may further include a cover configured to be positioned over the first layer which includes the dehydrated placental tissue.

In one aspect, the present disclosure provides a method of wound therapy, the method including applying the dressing of any embodiment herein to a tissue site. Additionally or alternatively, in some embodiments, the second layer may be configured as a wound contact layer. Additionally or alternatively, in some embodiments, the therapy is negative pressure wound therapy. Additionally or alternatively, in some embodiments, the method may further include sealing the dressing to tissue surrounding the tissue site to form a sealed space. Additionally or alternatively, in some embodiments, sealing the dressing to tissue surrounding the tissue site may include sealing a cover to the tissue surrounding the tissue site. Additionally or alternatively, in some embodiments, the method may further include fluidly coupling a negative-pressure source to the sealed space, and operating the negative-pressure source to generate a negative pressure in the sealed space.

In one aspect, the present disclosure provides a dressing that includes a first layer and a second layer, and wherein the first layer includes an effective amount of dehydrated placental tissue, and wherein the second layer includes a collagen and an oxidized regenerated cellulose (ORC), and wherein the second layer includes about 50% to about 60% collagen by weight, and wherein the second layer includes about 40% to about 50% ORC by weight.

Additionally or alternatively, in some embodiments, the second layer may include about 50%, about 51%, about 52%, about 53%, about 54%, about 55%, about 56%, about 57%, about 58%, about 59%, about 60%, or any range including and/or in between any two of these values, collagen by weight.

Additionally or alternatively, in some embodiments, the second layer may include about 40%, about 41%, about 42%, about 43%, about 44%, about 45%, about 46%, about 47%, about 48%, about 49%, about 50%, or any range including and/or in between any two of these values, ORC by weight.

In some embodiments, dehydrated placental tissue comprises amniotic membrane tissue, chorion tissue, or a combination thereof.

In some embodiments, the second layer is in a freeze-dried form. In some embodiments, the second layer is in the form of a film. In some embodiments, the second layer is in the form of a sponge.

In some embodiments, the second layer is configured as a wound contact layer.

In some embodiments, the second layer may include an antimicrobial agent. Additionally or alternatively, in some embodiments, the antimicrobial agent may be one or more of tetracycline, penicillins, terramycins, erythromycin, bacitracin, neomycin, polymycin B, mupirocin, clindamycin, colloidal silver, silver sulfadiazine, chlorhexidine, povidone iodine, triclosan, sucralfate, quaternary ammonium salts, pharmaceutically acceptable silver salts, or any combination thereof.

In some embodiments, the second layer may include silver and at least a portion of the silver may be present in a complex with the ORC (silver-ORC).

In some embodiments, the second layer may include an antioxidant. Additionally or alternatively, in some embodiments, the antioxidant may be one or more of anthocyanins, astaxanthin, bilirubin, canthaxanthin, capsaicin, citric acid, curcumin, coenzyme Q10, eugenol, flavanols, flavonolignans, flavanones, flavones, flavonols, iodide, isoflavone phytoestrogens, lutein, lycopene, manganese, melatonin, N-acetylcysteine, oxalic acid, phenolic acids, phytic acid, R-α-lipoic acid, stilbenoids, tocopherol, tocotrienol, vitamin A, vitamin C, vitamin E, xanthones, zeaxanthin, α-carotene, β-carotene, or any combination thereof.

In some embodiments, the dressing may further include a cover configured to be positioned over the first layer which includes the dehydrated placental tissue.

In one aspect, the present disclosure provides a method of wound therapy, the method including applying the dressing of any embodiment herein to a tissue site. Additionally or alternatively, in some embodiments, the second layer may be configured as a wound contact layer. Additionally or alternatively, in some embodiments, the therapy is negative pressure wound therapy. Additionally or alternatively, in some embodiments, the method may further include sealing the dressing to tissue surrounding the tissue site to form a sealed space. Additionally or alternatively, in some embodiments, sealing the dressing to tissue surrounding the tissue site may include sealing a cover to the tissue surrounding the tissue site. Additionally or alternatively, in some embodiments, the method may further include fluidly coupling a negative-pressure source to the sealed space, and operating the negative-pressure source to generate a negative pressure in the sealed space.

In another aspect, the present disclosure provides a method for treating a wound in a subject in need thereof, comprising administering to the wound a wound dressing of any embodiment disclosed herein. Additionally or alternatively, in some embodiments, the wound is an acute wound or a chronic wound. Additionally or alternatively in some embodiments, the wound dressing is applied directly to the wound.

In another aspect, the present disclosure provides a method for making a wound dressing, the method comprising providing dehydrated placental tissue, a collagen, and an oxidized regenerated cellulose (ORC), optionally wherein the dehydrated placental tissue, the collagen, and the ORC are present in a single layer. Additionally or alternatively, in some embodiments, the dressing is in the form of a sponge. Additionally or alternatively, in some embodiments, the dressing is in the form of a film.

In another aspect, the present disclosure provides a method for making a wound dressing, the method comprising providing a first layer and a second layer, wherein the first layer comprises an effective amount of dehydrated placental tissue, wherein the second layer comprises a collagen and an oxidized regenerated cellulose (ORC), wherein the second layer comprises about 50% to about 60% collagen by weight, and wherein the second layer comprise about 40% to about 50% ORC by weight. Additionally or alternatively, in some embodiments, the dressing is in the form of a sponge. Additionally or alternatively, in some embodiments, the dressing is in the form of a film. Additionally or alternatively, in some embodiments, the second layer includes at least one plasticizer. Additionally or alternatively, in some embodiments, the at least one plasticizer may include an acetylated monoglyceride, an alkyl citrate, methyl ricinoleate, glycerol, and a combination of any two or more thereof. Additionally or alternatively, in some embodiments, the alkyl citrate may include triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate, trimethyl citrate, or a combination of any two or more thereof.

Also provided herein are kits comprising the wound dressings of any embodiments disclosed herein and instructions for use.

### DRAWINGS

FIG. 1 is a perspective view of a dressing that can be used to treat tissue in accordance with this specification.

FIG. 2 is a perspective view of a dressing that can be used to treat tissue in accordance with this specification.

FIG. 3 is a perspective view of a dressing that can be used to treat tissue in accordance with this specification.

FIG. 4 is a simplified schematic diagram of an example embodiment of a negative pressure therapy system including the dressing of FIG. 1.

The accompanying drawings are intended to illustrate the general characteristics of certain example embodiments. The drawings may not precisely reflect the characteristics of any given embodiment, and are not intended to define or limit the scope of claimed technology.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

### Definitions

The definitions of certain terms as used in this specification are provided below. Unless defined otherwise, all technical and scientific terms used herein generally have the same meaning as commonly understood by one of ordinary skill in the art to which this present technology belongs.

The following terms are used throughout as defined below.

As used herein and in the appended claims, singular articles such as “a”, “an”, and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, the terms “contain”, “contains”, or “containing” in the context of describing the elements (especially in the context of the following claims) are to be construed as comprising or including the elements being described herein.

As used herein, the term “effective amount” refers to a quantity sufficient to achieve a desired therapeutic effect, e.g., an amount which results in the decrease in a wound described herein or one or more signs or symptoms associated with a wound described herein. In the context of therapeutic applications, the dressing administered to the subject will vary depending on the composition, the degree, type, and severity of the wound and on the characteristics of the individual.

As used herein, the terms “individual”, “patient”, or “subject” can be an individual organism, a vertebrate, a mammal, or a human. In some embodiments, the individual, patient or subject is a human.

As used herein, the terms “moisture vapor transmission rate” and “MVTR” will be understood by persons of ordinary skill in the art as a measure of the passage of water vapor through a substance of a given unit area and unit time. The most common international unit for the MVTR is g/m²/day, wherein 1 day=24 hr.

As understood by one of ordinary skill in the art, “molecular weight” (also known as “relative molar mass”) is a dimensionless quantity that can be converted to molar mass by multiplying by 1 gram/mole—for example, collagen with a weight-average molecular weight of 5,000 has a weight-average molar mass of 5,000 g/mol.

“Treating” or “treatment” as used herein covers the treatment of a wound described herein, in a subject, such as a human, and includes: (i) inhibiting a wound, i.e., arresting its development; (ii) relieving a wound, i.e., causing regression of the wound; (iii) slowing progression of the wound; and/or (iv) inhibiting, relieving, or slowing progression of one or more symptoms of the wound. In some embodiments, treatment means that the symptoms associated with the wound are, e.g., alleviated, reduced, cured, or placed in a state of remission.

It is also to be appreciated that the various modes of treatment of wounds as described herein are intended to mean “substantial,” which includes total but also less than total treatment, and wherein some biologically or medically relevant result is achieved. The treatment may be a continuous prolonged treatment for a chronic wound or a single, or several administrations for the treatment of an acute wound.

### The Dressing

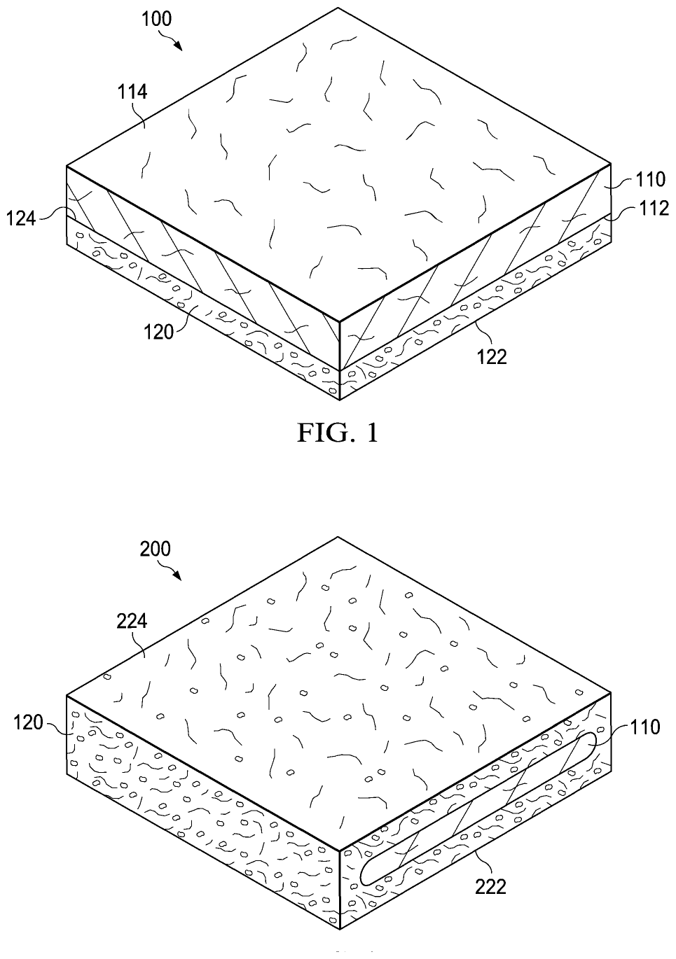

FIG. 1 is a perspective view of a dressing 100 that can be used to treat a tissue site in accordance with this specification.

The term “tissue site” is intended to broadly refer to a wound, defect, or other treatment target located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue.

The compositions, dressings, systems, and the methods described herein may provide significant advantages. For example, when employed in the context of a tissue treatment regime, the disclosed dressings may demonstrate improved therapeutic efficacy in comparison to conventional dehydrated amniotic membrane products. Without wishing to be bound by theory, it is believed that the disclosed dressings may advantageously reduce protease activity and wound bioburden while also providing a wound interface. For example, the dressings may advantageously promote cellular and vascular invasion into the wound space, provide a scaffold for wound-healing, provide extracellular matrix proteins and/or signaling molecules, or combinations thereof.

Without wishing to be bound by theory, it is believed that elevated protease activity may be destructive or detrimental to wound healing, for example, via the degradation of extracellular matrix proteins or growth factors. Additionally, the presence of elevated protease activity may also increase bioburden, thereby leading to decreased wound-healing. Further, elevated protease activity and increased bioburden may negatively impact the efficacy of such dehydrated amniotic membrane dressings. In some embodiments, the collagen/ORC component may be effective to modulate the activity of destructive enzymes such as elastase and matrix metalloproteinase (MMP) and neutrophil elastase. As such, the capability to use the disclosed dressings to modulate protease activity may be beneficial to wound healing.

In any embodiment disclosed herein, the dressing 100 may include a placental tissue component 110 and a collagen/OCR component 120.

Placental Tissue Component

In any embodiment disclosed herein, the placental tissue component 110 may comprise any suitable tissue or other collection of cells derived from placenta that may be provided to a tissue site to aid in wound-healing. Additionally or alternatively, in some embodiments, the placental tissue component 110 may be characterized as an allograft. The term “allograft” is intended to broadly refer to any tissue or other collection of cells whether viable or non-viable (e.g., living or non-living), derived from a genetically non-identical donor of the same species with respect to the intended recipient, for example, a generally non-identical human.

In any embodiment disclosed herein, the placental tissue component 110 may comprise at least a portion of amniotic membrane. The term “amniotic membrane” generally refers to the innermost layer of the placenta lining the amniotic cavity and includes multiple membrane layers that generally form the amniotic sac. Of these membranous layers, the innermost membrane is the amnion and the outermost membrane is the chorion. Additionally or alternatively, in some embodiments, the placental tissue component 110 may comprise amniotic membrane tissue, chorion tissue, or a combination thereof.

In any embodiment disclosed herein, the placental tissue component 110 may be dehydrated, for example, such that the placental tissue component 110 may be substantially free of water. Additionally or alternatively, in some embodiments, the placental tissue component 110 may contain 5% or less, 4.5% or less, 4% or less, 3.5% or less, 3% or less, 2.5% or less, 2% or less, 1.5% or less, 1% or less, 0.5% or less, or 0.1% or less of water, by weight of the placental tissue component. In any embodiment disclosed herein, the placental tissue component 110 may be freeze-dried. An example of the placental tissue component 110 is a dehydrated human amniotic membrane (DHAM). A non-limiting example of a commercially-available product that may be employed as the placental tissue component 110 is the EpiFix® dehydrated Human Amnion/Chorion Membrane (DHACM) allograft, commercially available from MiMedx® in Marietta, Ga.

In any embodiment herein, the placental tissue component may include dehydrated placental tissue. Additionally or alternatively, in some embodiments, the dehydrated placental tissue may be obtained from a single subject, patient, or placenta, or from a plurality of subjects, patients, or placentas. Additionally or alternatively, in some embodiments, the dressings of the present technology may include any number of dehydrated placenta cells. For example, a dressing of the present technology may include about 1×10⁵ cells, about 5×10⁵ cells, about 1×10⁶ cells, about 5×10⁶ cells, about 1×10⁷ cells, about 5×10⁷ cells, about 1×10⁸ cells, about 5×10⁸ cells, about 1×10⁹ cells, about 5×10⁹ cells, about 1×10¹⁰ cells, about 5×10¹⁰ cells, about 1×10¹¹ cells, or more placenta cells.

Collagen/ORC Component—Structural Protein (Collagen)

In any embodiment disclosed herein, the compositions and dressings described herein may further comprise a collagen/ORC component 120. In any embodiment disclosed herein, the collagen present may be obtained from any natural source. Additionally or alternatively, in some embodiments, the collagen may be Type I, II or III collagen, or a chemically-modified collagen, for example an atelocollagen obtained by removing the immunogenic telopeptides from natural collagen. Additionally or alternatively, in some embodiments, the collagen may also comprise solubilized collagen or soluble collagen fragments, for example, having a molecular weight in the range from about 5,000 to about 100,000, or from about 5,000 to about 50,000. Additionally or alternatively, in some embodiments, the collagen may also comprise solubilized collagen or soluble collagen fragments, for example, having a molecular weight in the range from about 5,000, about 6,000, about 7,000, about 8,000, about 9,000, about 10,000, about 12,000, about 14,000, about 16,000, about 18,000, about 20,000, about 22,000, about 24,000, about 26,000, about 28,000, about 30,000, about 32,000, about 34,000, about 36,000, about 38,000, about 40,000, about 42,000, about 44,000, about 46,000, about 48,000, about 50,000, about 52,000, about 54,000, about 56,000, about 58,000, about 60,000, about 62,000, about 64,000, about 66,000, about 68,000, about 70,000, about 72,000, about 74,000, about 76,000, about 78,000, about 80,000, about 82,000, about 84,000, about 86,000, about 88,000, about 90,000, about 92,000, about 94,000, about 96,000, about 98,000, about 100,000, or any range including and/or in between any two of the preceding values. Additionally or alternatively, in some embodiments, the solubilized collagen or soluble collagen fragments may be obtained by pepsin treatment of a natural collagen. Additionally or alternatively, in some embodiments, the collagen may be obtained from bovine corium that has been rendered largely free of non-collagenous components, for example, including fat, non-collagenous proteins, polysaccharides, and other carbohydrates, as described in U.S. Pat. Nos. 4,614,794, 4,320,201, 6,309,454, 8,461,410, and EP Patent 1758638, each incorporated by reference herein in their entirety.

In any embodiment disclosed herein, the collagen/ORC component 120 may comprise about 1% to about 90% collagen, or about 20% to about 70%, or about 40% to about 65%, or about 50% to about 60% collagen, by weight of the collagen/ORC component 120. Additionally or alternatively, in some embodiments, the collagen/ORC component 120 may comprise about 1% collagen, about 2% collagen, about 3% collagen, about 4% collagen, about 5% collagen, about 6% collagen, about 7% collagen, about 8% collagen, about 9% collagen, about 10% collagen, about 11% collagen, about 12% collagen, about 13% collagen, about 14% collagen, about 15% collagen, about 16% collagen, about 17% collagen, about 18% collagen, about 19% collagen, about 20% collagen, about 22% collagen, about 24% collagen, about 26% collagen, about 28% collagen, about 30% collagen, about 32% collagen, about 34% collagen, about 36% collagen, about 38% collagen, about 40% collagen, about 42% collagen, about 44% collagen, about 46% collagen, about 48% collagen, about 50% collagen, about 52% collagen, about 54% collagen, about 56% collagen, about 58% collagen, about 60% collagen, about 62% collagen, about 64% collagen, about 66% collagen, about 68% collagen, about 70% collagen, about 72% collagen, about 74% collagen, about 76% collagen, about 78% collagen, about 80% collagen, about 82% collagen, about 84% collagen, about 86% collagen, about 88% collagen, about 90% collagen, or any range including and/or in between any two of the preceding values, by weight of the collagen/ORC component 120.

In any embodiment disclosed herein, the collagen/ORC component 120 may further comprise a structural protein in addition to collagen. Examples of additional structural proteins may include, but are not limited to, fibronectin, fibrin, laminin, elastin, gelatins, and mixtures thereof.

Collagen/ORC Component—ORC

In any embodiment disclosed herein, at least a portion of the ORC present may be produced by the oxidation of cellulose, for example with dinitrogen tetroxide. Not intending to be bound by theory, this process may convert primary alcohol groups on the saccharide residues to carboxylic acid groups forming uronic acid residues, for example, within the cellulose chain. The oxidation may not proceed with complete selectivity, and as a result hydroxyl groups on carbons 2 and 3 may be converted to the keto form. These ketone units may introduce an alkali labile link, which at pH 7 or higher initiates the decomposition of the polymer via formation of a lactone and sugar ring cleavage. As a result, oxidized cellulose is biodegradable and bioabsorbable under physiological conditions.

Additionally or alternatively, in some embodiments, at least a portion of the ORC present may be prepared by oxidation of a regenerated cellulose, such as rayon. The ORC may be manufactured by the process described in U.S. Pat. No. 3,122,479, which is incorporated herein by reference in its entirety. ORC is available with varying degrees of oxidation and hence rates of degradation. Additionally or alternatively, in some embodiments, the ORC may be in the form of water-soluble, low molecular weight fragments, for example, obtained by alkali hydrolysis of ORC.

In any embodiment disclosed herein, the ORC present may be used in a variety of physical forms, including particles, fibers, a sheet, sponge, or fabrics. Additionally or alternatively, in some embodiments, the ORC may be in the form of particles, such as fiber particles or powder particles, for example dispersed in a suitable solid or semisolid topical medicament vehicle. Additionally or alternatively, in some embodiments, the ORC comprises ORC fibers. Additionally or alternatively, in some embodiments, the ORC fibers may have a volume fraction such that at least 80% of the fibers have lengths in the range from about 5 μm to about 1000 μm, or in some more particular embodiments, from about 250 μm to about 450 μm. Additionally or alternatively, in some embodiments, the ORC may include fiber lengths of about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 22 μm, about 24 μm, about 26 μm, about 28 μm, about 30 μm, about 32 μm, about 34 μm, about 36 μm, about 38 μm, about 40 μm, about 42 μm, about 44 μm, about 46 μm, about 48 μm, about 50 μm, about 55 μm, about 60 μm, about 65 μm, about 70 μm, about 75 μm, about 80 μm, about 85 μm, about 90 μm, about 95 μm, about 100 μm, about 110 μm, about 120 μm, about 130 μm, about 140 μm, about 150 μm, about 160 μm, about 170 μm, about 180 μm, about 190 μm, about 200 μm, about 220 μm, about 230 μm, about 240 μm, about 250 μm, about 260 μm, about 280 μm, about 300 μm, about 320 μm, about 340 μm, about 360 μm, about 380 Inn, about 400 Inn, about 420 μm, about 440 μm, about 460 μm, about 480 μm, about 500 μm, about 550 μm, about 600 μm, about 650 μm, about 700 μm, about 750 μm, about 800 μm, about 850 μm, about 900 μm, about 950 μm, about 1,000 μm, or any range including and/or in between any two of the preceding values. Additionally or alternatively, in some embodiments, a desired size distribution can be achieved, for example, by milling an ORC cloth, followed by sieving the milled powder to remove fibers outside the range. Such fabrics may include woven, non-woven and knitted fabrics.

In any embodiment disclosed herein, the collagen/ORC component 120 may comprise about 10% to about 98% ORC, or about 30% to about 95%, or about 40% to about 50% ORC, by weight of the collagen/ORC component 120. Additionally or alternatively, in some embodiments, the collagen/ORC component 120 may comprise about 10% ORC, about 11% ORC, about 12% ORC, about 13% ORC, about 14% ORC, about 15% ORC, about 16% ORC, about 17% ORC, about 18% ORC, about 19% ORC, about 20% ORC, about 22% ORC, about 24% ORC, about 26% ORC, about 28% ORC, about 30% ORC, about 32% ORC, about 34% ORC, about 36% ORC, about 38% ORC, about 40% ORC, about 42% ORC, about 44% ORC, about 46% ORC, about 48% ORC, about 50% ORC, about 52% ORC, about 54% ORC, about 56% ORC, about 58% ORC, about 60% ORC, about 62% ORC, about 64% ORC, about 66% ORC, about 68% ORC, about 70% ORC, about 72% ORC, about 74% ORC, about 76% ORC, about 78% ORC, about 80% ORC, about 82% ORC, about 84% ORC, about 86% ORC, about 88% ORC, about 90% ORC, about 92% ORC, about 94% ORC, about 96% ORC, about 98% ORC, or any range including and/or in between any two of the preceding values, by weight of the collagen/ORC component 120.

Additionally or alternatively, in some embodiments, the collagen/ORC component 120 may comprise about 40% to about 65%, or about 50% to about 60% collagen, and about 30% to about 95%, or about 40% to about 50% ORC, by weight of the collagen/ORC component 120.

Optional Materials

In any embodiment disclosed herein, the placental tissue component 110, the collagen/ORC component 120, or both may optionally further comprise one or more suitable additives, such as those that may be effective to promote wound-healing. Examples of such additives may include active materials, antimicrobial agents, preservatives, stabilizing agents, plasticizers, strengthening materials, dyestuffs, and combinations thereof.

In any embodiment disclosed herein, examples of an active material may include, but are not limited to, growth factors, such as platelet derived growth factor (PDGF), transforming growth factor beta (TGFβ), fibroblast growth factors (FGFs), epidermal growth factor (EGF), or mixtures thereof. Additionally or alternatively, in some embodiments, the fibroblast growth factors (FGFs) may be one or more of fibroblast growth factor 1 (FGF1), fibroblast growth factor 2 (FGF2), fibroblast growth factor 3 (FGF3), fibroblast growth factor 4 (FGF4), fibroblast growth factor 5 (FGF5), fibroblast growth factor 6 (FGF6), fibroblast growth factor 7/keratinocyte growth factor (FGF7/KGF), fibroblast growth factor 8 (FGF8), fibroblast growth factor 9 (FGF9), fibroblast growth factor 10/keratinocyte growth factor 2 (FGF10/KGF2), fibroblast growth factor 11 (FGF11), fibroblast growth factor 12 (FGF12), fibroblast growth factor 13 (FGF13), fibroblast growth factor 14 (FGF14), fibroblast growth factor 15 (FGF15), fibroblast growth factor 16 (FGF16), fibroblast growth factor 17 (FGF17), fibroblast growth factor 18 (FGF18), fibroblast growth factor 19 (FGF19), fibroblast growth factor 20 (FGF20), fibroblast growth factor 21 (FGF21), fibroblast growth factor 22 (FGF22), fibroblast growth factor 23 (FGF23), or any combination thereof. Additionally or alternatively, in some embodiments, examples of an active material may include, but are not limited to, nutrients, such as amino acids, vitamins, essential fatty acids; extracellular matrix proteins, and mixtures thereof.

In any embodiment disclosed herein, examples of an active material may include, but are not limited to, non-steroidal anti-inflammatory drugs (e.g. acetaminophen), steroids, antimicrobial agents (e.g. penicillins or streptomycins), antiseptics (e.g. chlorhexidine), and combinations thereof. Additionally or alternatively, in some embodiments, such active materials can be present at a level from about 0.1% to about 10%, or from about 1% to about 5%, by weight of the collagen/ORC component 120. Additionally or alternatively, in some embodiments, such active materials can be present at a level from about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.2%, about 2.4%, about 2.6%, about 2.8%, about 3%, about 3.2%, about 3.4%, about 3.6%, about 3.8%, about 4%, about 4.2%, about 4.4%, about 4.6%, about 4.8%, about 5%, about 5.2%, about 5.4%, about 5.6%, about 5.8%, about 6%, about 6.2%, about 6.4%, about 6.6%, about 6.8%, about 7%, about 7.2%, about 7.4%, about 7.6%, about 7.8%, about 8%, about 8.2%, about 8.4%, about 8.6%, about 8.8%, about 9%, about 9.2%, about 9.4%, about 9.6%, about 9.8%, about 10%, or any range including and/or in between any two of the preceding values, by weight of the collagen/ORC component 120.

In any embodiment disclosed herein, the antimicrobial agents may comprise a safe and effective amount of poly(hexamethylene biguanide) (“PHMB”), which is also known as polyaminopropyl biguanid (“PAPB”) and polyhexanide, having the following general formula.

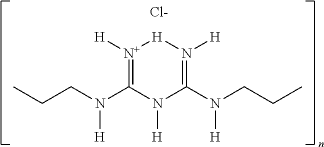

PHMB is a cationic broad spectrum antimicrobial agent. PHMB may be synthesized by a variety of methods, including polycondensation of sodium dicyanamide and hexamethylenediamine. In any embodiment disclosed herein, the collagen/ORC component 120 may comprise about 0.005% to about 0.025% PHMB, or about 0.007% to about 0.2%, or about 0.008% to about 0.012% PHMB, by weight of the collagen/ORC component 120. Additionally or alternatively, in some embodiments, the collagen/ORC component 120 may comprise about 0.005% PHMB, about 0.006% PHMB, about 0.007% PHMB, about 0.008% PHMB, about 0.009% PHMB, about 0.010% PHMB, about 0.011% PHMB, about 0.012% PHMB, about 0.013% PHMB, about 0.014% PHMB, about 0.015% PHMB, about 0.016% PHMB, about 0.017% PHMB, about 0.018% PHMB, about 0.019% PHMB, about 0.020% PHMB, about 0.022% PHMB, about 0.024% PHMB, about 0.026% PHMB, about 0.028% PHMB, about 0.030% PHMB, about 0.032% PHMB, about 0.034% PHMB, about 0.036% PHMB, about 0.038% PHMB, about 0.040% PHMB, about 0.042% PHMB, about 0.044% PHMB, about 0.046% PHMB, about 0.048% PHMB, about 0.050% PHMB, about 0.055% PHMB, about 0.060% PHMB, about 0.065% PHMB, about 0.070% PHMB, about 0.075% PHMB, about 0.080% PHMB, about 0.085% PHMB, about 0.090% PHMB, about 0.095% PHMB, about 0.1% PHMB, about 0.11% PHMB, about 0.12% PHMB, about 0.13% PHMB, about 0.14% PHMB, about 0.15% PHMB, about 0.16% PHMB, about 0.17% PHMB, about 0.18% PHMB, about 0.19% PHMB, about 0.2% PHMB, or any range including and/or in between any two of the preceding values, by weight of the collagen/ORC component 120. Additionally or alternatively, in some embodiments, the collagen/ORC component 120 may comprise about 0.01% PHMB, by weight of the collagen/ORC component 120.

In any embodiment disclosed herein, the collagen/ORC component 120 may comprise one or more antimicrobial agents. Additionally or alternatively, in some embodiments, the one or more antimicrobial agents may be one or more of tetracycline, penicillins, terramycins, erythromycin, bacitracin, neomycin, polymycin B, mupirocin, clindamycin, colloidal silver, silver sulfadiazine, chlorhexidine, povidone iodine, triclosan, sucralfate, quaternary ammonium salts, pharmaceutically acceptable silver salts, or any combination thereof.

In any embodiment disclosed herein, the solution may comprise an antioxidant. Additionally or alternatively, in some embodiments of the wound dressing disclosed herein, the solution may comprise about 0.001 wt % to about 5 wt % of the antioxidant. Additionally or alternatively, in some embodiments of the wound dressing disclosed herein, the antioxidant may comprise about 0.001 wt %, about 0.002 wt %, about 0.003 wt %, about 0.004 wt %, about 0.005 wt %, about 0.006 wt %, about 0.007 wt %, about 0.008 wt %, about 0.009 wt %, about 0.01 wt %, about 0.02 wt %, about 0.03 wt %, about 0.04 wt %, about 0.05 wt %, about 0.06 wt %, about 0.07 wt %, about 0.08 wt %, about 0.09 wt %, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, about 2 wt %, about 2.2 wt %, about 2.4 wt %, about 2.6 wt %, about 2.8 wt %, about 3 wt %, about 3.2 wt %, about 3.4 wt %, about 3.6 wt %, about 3.8 wt %, about 4 wt %, about 4.2 wt %, about 4.4 wt %, about 4.6 wt %, about 4.8 wt %, about 5 wt %, or any range including and/or in between any two of these values. Additionally or alternatively, in some embodiments of the wound dressing disclosed herein, the antioxidant may be one or more of anthocyanins, astaxanthin, bilirubin, canthaxanthin, capsaicin, citric acid, curcumin, coenzyme Q10, eugenol, flavanols, flavonolignans, flavanones, flavones, flavonols, iodide, isoflavone phytoestrogens, lutein, lycopene, manganese, melatonin, N-acetylcysteine, oxalic acid, phenolic acids, phytic acid, R-α-lipoic acid, stilbenoids, tocopherol, tocotrienol, vitamin A, vitamin C, vitamin E, xanthones, zeaxanthin, α-carotene, β-carotene, or any combination thereof.

In any embodiment of the wound dressing disclosed herein, the anthocyanins are selected from the group consisting of cyanidin, delphinidin, malvidin, pelargonidin, peonidin, petunidin, and mixtures thereof.

In any embodiment of the wound dressing disclosed herein, the flavanols are selected from the group consisting of catechin, epicatechin, theaflavin, thearubigins, gallocatechin, epigallocatechin, or any gallate ester thereof, and mixtures thereof.

In any embodiment of the wound dressing disclosed herein, the flavanones are selected from the group consisting of eriodictyol, hesperetin, naringenin, and mixtures thereof.

In any embodiment of the wound dressing disclosed herein, the flavones are selected from the group consisting of apigenin, luteolin, tangeritin, and mixtures thereof.

In any embodiment of the wound dressing disclosed herein, the flavonols are selected from the group consisting of isorhamnetin, kaempferol, myricetin, proanthocyanidins, quercetin, rutin, and mixtures thereof.

In any embodiment of the wound dressing disclosed herein, the isoflavone phytoestrogens are selected from the group consisting of daidzein, genistein, glycitein, and any combination thereof.

In any embodiment of the wound dressing disclosed herein, the phenolic acids are selected from the group consisting of chicoric acid, chlorogenic acid, cinnamic acid, ellagic acid, ellagitannins, gallic acid, gallotannins, rosmarinic acid, salicylic acid, or any ester thereof, and any combination thereof.

In any embodiment of the wound dressing disclosed herein, the stillbenoids are selected from the group consisting of resveratrol, pterostilbene, and any combination thereof.

In any embodiment disclosed herein, carboxymethylcellulose (CMC) may be present as a modifier, for example, which may modify one or more characteristics of the composition, for example, the rheological, absorbency, and other structural characteristics of the composition. Additionally or alternatively, in some embodiments, CMC may be present in the collagen/ORC component 120 at any level appropriate to result in the desired absorbency and rheological characteristics of the collagen/ORC component 120.

In any embodiment herein, examples of a strengthening material, which can improve the handling characteristics by decreasing susceptibility to tearing, may comprise non-gelling cellulose fibers. Non-gelling cellulose fibers may be substantially water insoluble and may be produced from cellulose that has not been chemically modified to increase water solubility (e.g., as contrasted from carboxymethyl cellulose or other cellulose ethers). Non-gelling cellulose fibers are commercially available, such as Tencel® fibers (sold by Lenzing AG). In any embodiment herein, such fibers may be processed from a commercially-available continuous length, by cutting into lengths that are about 0.5 cm to about 5 cm, or about 2 cm to about 3 cm in length. Additionally or alternatively, in some embodiments, the fibers may be processed from a commercially-available continuous length, by cutting into lengths that are about 0.5 cm, about 0.6 cm, about 0.7 cm, about 0.8 cm, about 0.9 cm, about 1 cm, about 1.1 cm, about 1.2 cm, about 1.3 cm, about 1.4 cm, about 1.5 cm, about 1.6 cm, about 1.7 cm, about 1.8 cm, about 2 cm, about 2.2 cm, about 2.4 cm, about 2.6 cm, about 2.8 cm, about 3 cm, about 3.2 cm, about 3.4 cm, about 3.6 cm, about 3.8 cm, about 4 cm, about 4.2 cm, about 4.4 cm, about 4.6 cm, about 4.8 cm, about 5 cm, or any range including and/or in between any two of the preceding values, in length. Additionally or alternatively, in some embodiments, the non-gelling cellulose fibers may be present in the composition at any level appropriate to result in the desired physical characteristics of the composition. Additionally or alternatively, in some embodiments, the non-gelling cellulose fibers may be present at a level about 1% to about 25% by weight of the collagen/ORC component 120, or about 5% to about 20%, or about 10% to about 15%, by weight of the collagen/ORC component 120. Additionally or alternatively, in some embodiments, the non-gelling cellulose fibers may be present at a level about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, or any range including and/or in between any two of the preceding values, by weight of the collagen/ORC component 120.

Additionally or alternatively, in some embodiments, the collagen/ORC component 120 may comprise silver in an amount from about 0.25% to about 2% by weight of the dressing. Additionally or alternatively, in some embodiments, the collagen/ORC component 120 may comprise silver in an amount from about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 1.1%, about 1.2%, about 1.3%, about 1.4%, about 1.5%, about 1.6%, about 1.7%, about 1.8%, about 1.9%, about 2%, or any range including and/or in between any two of the preceding values, by weight of the dressing. Suitable, non-limiting examples of a component comprising collagen and ORC include the PROMOGRAN™ Matrix Wound Dressing and the PROMOGRAN PRISMA™ Matrix, commercially available from Acelity L.P. in San Antonio, Tex. Additionally or alternatively, in some embodiments, at least a portion of the silver may be present in a complex with the ORC (silver-ORC).

In any embodiment disclosed herein, the collagen/ORC component 120 may include at least one plasticizer. Additionally or alternatively, in some embodiments, the collagen/ORC component 120 may include about 1 wt. % to about 10 wt. % of the at least one plasticizer. Additionally or alternatively, in some embodiments, the collagen/ORC component 120 may include about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.2 wt. %, about 2.4 wt. %, about 2.6 wt. %, about 2.8 wt. %, about 3 wt. %, about 3.2 wt. %, about 3.4 wt. %, about 3.6 wt. %, about 3.8 wt. %, about 4 wt. %, about 4.2 wt. %, about 4.4 wt. %, about 4.6 wt. %, about 4.8 wt. %, about 5 wt. %, about 5.2 wt. %, about 5.4 wt. %, about 5.6 wt. %, about 5.8 wt. %, about 6 wt. %, about 6.2 wt. %, about 6.4 wt. %, about 6.6 wt. %, about 6.8 wt. %, about 7 wt. %, about 7.2 wt. %, about 7.4 wt. %, about 7.6 wt. %, about 7.8 wt. %, about 8 wt. %, about 8.2 wt. %, about 8.4 wt. %, about 8.6 wt. %, about 8.8 wt. %, about 9 wt. %, about 9.2 wt. %, about 9.4 wt. %, about 9.6 wt. %, about 9.8 wt. %, about 10 wt. %, or any range including and/or in between any two of the preceding values, of the at least one plasticizer. Additionally or alternatively, in some embodiments, the at least one plasticizer may be an acetylated monoglyceride, an alkyl citrate, methyl ricinoleate, glycerol, or a combination of any two or more thereof. Additionally or alternatively, in some embodiments, the alkyl citrate is triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate, trimethyl citrate, or a combination of any two or more thereof.

Collagen/ORC Component Characteristics

In any embodiment disclosed herein, the collagen/ORC component 120 may be generally characterized as being biologically-active or as exhibiting biological activity. Additionally or alternatively, in some embodiments, the collagen/ORC component 120 may be characterized as exhibiting protease-modulating activity. Modulation of protease activity may include inhibition of destructive enzymes such as neutrophil elastase and matrix metalloproteinase (MMP). Additionally or alternatively, in some embodiments, the collagen/ORC component 120 may be effective to inhibit protease activity such that protease activity is decreased to less than about 75% of the protease activity that would be present if uninhibited, or to less than about 50%, or to less than about 40%, or to less than about 30% to less than about 20% of the protease activity that would be present if uninhibited. Additionally or alternatively, in some embodiments, the collagen/ORC component 120 may be effective to inhibit protease activity such that protease activity is decreased to less than about 75% to about less than 20% of the protease activity that would be present if uninhibited. Additionally or alternatively, in some embodiments, the collagen/ORC component 120 may be effective to inhibit protease activity such that protease activity is decreased to less than about 75%, less than about 70%, less than about 65%, less than about 60%, less than about 55%, less than about 50%, less than about 48%, less than about 46%, less than about 44%, less than about 42%, less than about 40%, less than about 38%, less than about 36%, less than about 34%, less than about 36%, less than about 34%, less than about 32%, less than about 30%, less than about 28%, less than about 26%, less than about 24%, less than about 22%, less than about 20%, or any range including and/or in between any two of the preceding values.

In any embodiment herein, the collagen/ORC component 120 may be characterized as being biodegradable or as exhibiting biodegradability. As used herein, “biodegradable” and “biodegradability” may refer to a characteristic of a material to at least partially break down upon exposure to physiological fluids or processes. Additionally or alternatively, in some embodiments, the collagen/ORC component 120 may disintegrate, degrade, or dissolve when contacted with an aqueous medium, such as water, blood, or wound exudate from a tissue site. Biodegradability may be a result of a chemical process or condition, a physical process or condition, or combinations thereof.

Additionally or alternatively, in some embodiments, the collagen/ORC component 120 may be characterized as being bioresorbable or as exhibiting bioresorbability. As used herein, “bioresorbable” and “bioresorbability” may refer to a characteristic of a material to be broken down into degradation products that may be assimilated at a tissue site so as to be eliminated by the body, for example via metabolism or excretion. Additionally or alternatively, in some embodiments the bioresorbable characteristics of the collagen/ORC component 120 may be such that at least a portion of the collagen/ORC component 120 may be eliminated from the tissue site to which it is applied by bioresorption.

For example, in some embodiments, the collagen/ORC component 120 may be configured to exhibit a particular proportion of disintegration, degradation, or dissolution within a particular time period. For instance, in various embodiments the collagen/ORC component 120 may be configured such that about 90% by weight, or about 95% by weight, or about 99% by weight, or about 100% by weight of the dressing 100 may be disintegrated, degraded, or dissolved with in a time period from about 24 hours to about 7 days from introduction into a physiological environment or when incubated with simulated physiological fluid at a temperature of about 37° C.

In any embodiment herein, the collagen/ORC component 120 includes a suitable structure, for example, the collagen/ORC component 120 may be in the form of a film, a sponge, or a combination thereof. Additionally or alternatively, in some embodiments, the collagen/ORC component 120 may be a freeze-dried form.

Dressing Layers

In any embodiment herein, the dressing 100 may comprise one or more layers which may be configured to interface with a tissue site. Additionally or alternatively, in some embodiments, the dressing 100 may be generally configured to be positioned adjacent to a tissue site. The dressing 100 may be configured to be in contact with a portion of a tissue site, substantially all of a tissue site, or a tissue site in its entirety. For example, if a tissue site is a wound, the dressing 100 may partially or completely fill the wound, or may be placed over the wound. Additionally or alternatively, in some embodiments, the collagen/ORC component 120 may be configured as a wound contact layer. Additionally or alternatively, in some embodiments, the dressing 100 may take many forms, and may have many sizes, shapes, or thicknesses depending on a variety of factors, such as the type of treatment being implemented or the nature and size of a tissue site. Additionally or alternatively, in some embodiments, the size and shape of the dressing 100 may be adapted to the contours of deep and irregular shaped tissue sites and/or may be configured so as to be adaptable to a given shape or contour. Additionally or alternatively, in some embodiments, any or all of the surfaces of the dressing 100 may comprise projections or an uneven, course, or jagged profile that can, for example, induce strains and stresses on a tissue site, for example, which may be effective to promote granulation at a tissue site.

In any embodiment disclosed herein, the placental tissue component 110 and the collagen/ORC component 120 may comprise separate and/or discernable layers. For example, in the embodiment of FIG. 1, the placental tissue component 110 and the collagen/ORC component 120 may each generally comprise a layer having a generally planar structure including two opposite-facing planar surfaces and a depth or thickness orthogonal to the planar surfaces. Additionally or alternatively, in some embodiments, the placental tissue component 110 may comprise a first surface 112 and a second surface 114. The first surface 112 may be configured to face a tissue site, and the second surface 114 may be opposite the first surface 112. Additionally or alternatively, in some embodiments, the collagen/ORC component 120 may comprise a first surface 122 and a second surface 124. The first surface 122 may be configured to face a tissue site, and the second surface 124 may be opposite the first surface 122. Additionally or alternatively, in some embodiments, the placental tissue component 110 may be present in a first layer. Additionally or alternatively, in some embodiments, the collagen/ORC component 120 may be present in a second layer.

In any embodiment disclosed herein, the surfaces of a layer formed by the placental tissue component 110, the collagen/ORC component 120, or both may have a surface area from about 1 cm² to about 400 cm², from about 2 cm² to about 200 cm², or from about 4 cm² to about 100 cm². Additionally or alternatively, in some embodiments, the surfaces of a layer formed by the placental tissue component 110, the collagen/ORC component 120, or both may have a surface area from about 1 cm², about 2 cm², about 3 cm², about 4 cm², about 5 cm², about 6 cm², about 7 cm², about 8 cm², about 9 cm², about 10 cm², about 11 cm², about 12 cm², about 13 cm², about 14 cm², about 15 cm², about 16 cm², about 17 cm², about 18 cm², about 19 cm², about 20 cm², about 22 cm², about 24 cm², about 26 cm², about 28 cm², about 30 cm², about 32 cm², about 34 cm², about 36 cm², about 38 cm², about 40 cm², about 42 cm², about 44 cm², about 46 cm², about 48 cm², about 50 cm², about 52 cm², about 54 cm², about 56 cm², about 58 cm², about 60 cm², about 62 cm², about 64 cm², about 66 cm², about 68 cm², about 70 cm², about 72 cm², about 74 cm², about 76 cm², about 78 cm², about 80 cm², about 82 cm², about 84 cm², about 86 cm², about 88 cm², about 90 cm², about 92 cm², about 94 cm², about 96 cm², about 98 cm², about 100 cm², about 110 cm², about 120 cm², about 130 cm², about 140 cm², about 150 cm², about 160 cm², about 170 cm², about 180 cm², about 190 cm², about 200 cm², about 220 cm², about 240 cm², about 260 cm², about 280 cm², about 300 cm², about 320 cm², about 340 cm², about 360 cm², about 380 cm², about 400 cm², or any range including and/or in between any two of the preceding values. Additionally or alternatively, in some embodiments, surfaces of the layer formed by the placental tissue component 110, the collagen/ORC component 120, or both may have any suitable shape, examples of which include but are not limited to, triangles, squares, rectangles, ellipses, circles, ovals, and various polygons having four, five, six, seven, eight, or more sides. The shape and area of the surfaces of a layer formed by the placental tissue component 110, the collagen/ORC component 120, or both may be customized to the location and type of tissue site onto which the dressing 100 is to be applied.

In any embodiment disclosed herein, the placental tissue component 110 and the collagen/ORC component 120 comprise separate and/or discernable layers. Additionally or alternatively, in some embodiments, the collagen/ORC component 120 may be in contact with a surface of the placental tissue component 110. In the embodiment of FIG. 1, the collagen/ORC component 120 may be in contact with a surface of the placental tissue component 110, for example, such that the first surface 112 of the placental tissue component is in contact with the second surface 124 of the collagen/ORC component 120.

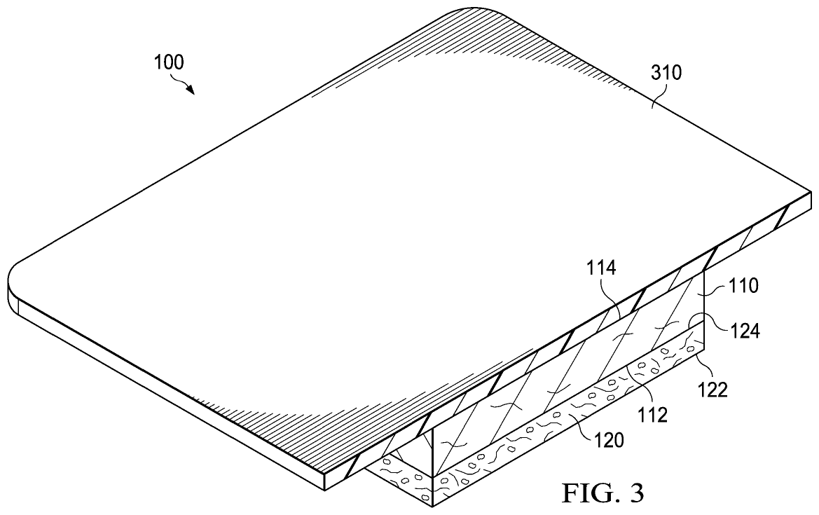

Additionally or alternatively, in some embodiments, the placental tissue component 110 may be incorporated within the collagen/ORC component 120. For example, in the embodiment of FIG. 2, a dressing 200 may include a single layer comprising the placental tissue component 110 incorporated within the collagen/ORC component 120. The collagen/ORC component 120 may partially, substantially, or entirely cover the placental tissue component 110 or one or more surfaces thereof. In the embodiment of FIG. 2, the dressing 200 may comprise a first surface 222 and a second surface 224. The first surface 222 may be configured to face a tissue site, and the second surface 224 may be opposite the first surface 222.

Additional Layers

In any embodiment disclosed herein, the dressing 100 may comprise one or more additional layers. Additionally or alternatively, in some embodiments, additional layers may perform any of a variety of functions including, for example, adhering the dressing 100 to a tissue site or to surrounding tissue, increasing structural rigidity, protecting a tissue site from moisture or other materials in the external environment, protecting a tissue surface, delivering one or more active materials to a tissue surface, or a combination thereof. Additionally or alternatively, in some embodiments, additional layers may conform to a surface of a tissue site, to surrounding tissue, or both. For example, an additional layer may be capable of bending or deforming such that a surface of the dressing 100 may be in substantial contact with a tissue site.

For example, in the embodiment of FIG. 3, the dressing 100 further comprises a cover 310. The cover 310 may have a first surface configured to face a tissue site, and a second surface opposite the first surface. Additionally or alternatively, in some embodiments, for example, the second surface 114 of the placental tissue component 110 may be in contact with and adhered to the first surface of the cover 310.

In any embodiment disclosed herein, the cover 310 may generally be configured to provide a bacterial barrier and protection from physical trauma. Additionally or alternatively, in some embodiments, the cover 310 may also be constructed from a material that can reduce evaporative losses and provide a fluid seal between two components or two environments, such as between a therapeutic environment and a local external environment. Additionally or alternatively, in some embodiments, the cover 310 may be, for example, an elastomeric film or membrane that can provide a seal at a tissue site. Additionally or alternatively, in some embodiments, the cover 310 may have a high moisture-vapor transmission rate (MVTR). For example, in such an embodiment, the MVTR may be at least 300 g/m² per twenty-four hours. Additionally or alternatively, in some embodiments, the cover 310 may be formed from a suitable polymer. For example, the cover 310 may comprise a polymer drape, such as a polyurethane film, that may be permeable to water vapor but generally impermeable to liquid. Additionally or alternatively, in some embodiments, the cover 310 may have a thickness in the range of about from 25 to about 50 microns. Additionally or alternatively, in some embodiments, the cover 310 may have a thickness in the range of about 25 microns, about 26 microns, about 27 microns, about 28 microns, about 29 microns, about 30 microns, about 31 microns, about 32 microns, about 33 microns, about 34 microns, about 35 microns, about 36 microns, about 37 microns, about 38 microns, about 39 microns, about 40 microns, about 41 microns, about 42 microns, about 43 microns, about 44 microns, about 45 microns, about 46 microns, about 47 microns, about 48 microns, about 49 microns, about 50 microns, or any range including and/or in between any two of the preceding values.

In any embodiment disclosed herein, an attachment device may be used to attach the cover 310 to an attachment surface, such as undamaged epidermis, a gasket, or another cover. The attachment device may take many forms. Additionally or alternatively, in some embodiments, an attachment device may be a medically-acceptable, pressure-sensitive adhesive configured to bond the cover 310 to epidermis around a tissue site. Additionally or alternatively, in some embodiments, some or all of the cover 310 may be coated with an adhesive, such as an acrylic adhesive, which may have a coating weight of about 25 to about 65 grams per square meter (g.s.m.). Additionally or alternatively, in some embodiments, some or all of the cover 310 may be coated with an adhesive which may have a coating weight of about 25 g.s.m., about 26 g.s.m., about 27 g.s.m., about 28 g.s.m., about 29 g.s.m., about 30 g.s.m., about 31 g.s.m., about 32 g.s.m., about 33 g.s.m., about 34 g.s.m., about 35 g.s.m., about 36 g.s.m., about 37 g.s.m., about 38 g.s.m., about 39 g.s.m., about 40 g.s.m., about 41 g.s.m., about 42 g.s.m., about 43 g.s.m., about 44 g.s.m., about 45 g.s.m., about 46 g.s.m., about 47 g.s.m., about 48 g.s.m., about 49 g.s.m., about 50 g.s.m., about 51 g.s.m., about 52 g.s.m., about 53 g.s.m., about 54 g.s.m., about 55 g.s.m., about 56 g.s.m., about 57 g.s.m., about 58 g.s.m., about 59 g.s.m., about 60 g.s.m., about 61 g.s.m., about 62 g.s.m., about 63 g.s.m., about 64 g.s.m., about 65 g.s.m., or any range including and/or in between any two of the preceding values. Thicker adhesives, or combinations of adhesives, may be applied in some embodiments, for example, to improve the seal. Additionally or alternatively, in some embodiments, an attachment device may include a double-sided tape, a paste, a hydrocolloid, a hydrogel, a silicone gel, or an organogel.

Additionally or alternatively, in some embodiments, the dressing may comprise a secondary layer. A secondary layer may comprise fluid pathways interconnected so as to improve distribution or collection of fluids. Additionally or alternatively, in some embodiments, a secondary layer may comprise or consist essentially of a porous material. Examples of suitable porous material may include cellular foam, including open-cell foam such as reticulated foam, porous tissue collections, and other porous material such as gauze or felted mat that generally include pores, edges, and/or walls adapted to form interconnected fluid pathways (e.g., channels). Additionally or alternatively, in some embodiments, a secondary layer may comprise or consist essentially of reticulated polyurethane foam.

In any embodiment disclosed herein, a secondary layer may be characterized as exhibiting absorbency. For example, a secondary layer may exhibit an absorbency of at least 3 g saline/g, or at least 5 g saline/g, or from about 8 g saline/g to about 20 g saline/g. In some embodiments, a secondary layer may be hydrophilic. Additionally or alternatively, in some embodiments, a secondary layer may exhibit an absorbency of about 8 g saline/g, about 9 g saline/g, about 10 g saline/g, about 11 g saline/g, about 12 g saline/g, about 13 g saline/g, about 14 g saline/g, about 15 g saline/g, about 16 g saline/g, about 17 g saline/g, about 18 g saline/g, about 19 g saline/g, about 20 g saline/g, or any range including and/or in between any two of the preceding values. Additionally or alternatively, in some embodiments, a secondary layer may be hydrophilic. Additionally or alternatively, in some embodiments, the secondary layer may also absorb or wick fluid away from the placental tissue component 110 and the collagen/ORC component 120. The wicking properties of a secondary layer may draw fluid away from the placental tissue component 110 and the collagen/ORC component 120 by capillary flow or other wicking mechanisms. Exemplary hydrophilic foams include, but are not limited to a polyvinyl alcohol, open-cell foam. Additionally or alternatively, in some embodiments, other hydrophilic foams may include those made from polyether. Other foams that may exhibit hydrophilic characteristics include hydrophobic foams that have been treated or coated to provide hydrophilicity.

Methods of Making the Dressing

In any embodiment disclosed herein, a method for preparing a dressing may comprise providing a placental tissue component. Additionally or alternatively, in some embodiments, a suitable commercially-available product, such as the EpiFix® dehydrated Human Amnion/Chorion Membrane (dHACM) allograft, commercially available from MiMedx® in Marietta, Ga., may be used as the placental tissue component. Suitable methods of harvesting, processing, and preparing a placental material suitable for use as the placental tissue component are disclosed in U.S. Pat. No. 8,460,715, the entirety of which is incorporated herein by reference. Additionally or alternatively, in some embodiments, the placental tissue component may be derived from placental tissue collected during a Cesarean section birth. Additionally or alternatively in some embodiments, the placental tissue may be subjected to a screening process, for example, to determine the presence of antibodies that may indicate that the tissue is unfit for use as the placental tissue component 110. Additionally or alternatively in some embodiments, the placental tissue may undergo further processing, for example, decontamination, sizing, and evaluation. Additionally or alternatively in some embodiments, the amnion and chorion layers of the placental tissue may be separated.

In any embodiment disclosed herein, the processed placental tissue may be dehydrated. For example, the placental tissue may be dehydrated in a non-vacuum oven or an incubator for a suitable time, such as from about 30 minutes to 120 minutes at a temperature from about 35 to 50 degrees Celsius. Additionally or alternatively, in some embodiments, the placental tissue may be dehydrated in a non-vacuum oven or an incubator for about 30 minutes, about 32 minutes, about 34 minutes, about 36 minutes, about 38 minutes, about 40 minutes, about 42 minutes, about 44 minutes, about 46 minutes, about 48 minutes, about 50 minutes, about 52 minutes, about 54 minutes, about 56 minutes, about 58 minutes, about 60 minutes, about 62 minutes, about 64 minutes, about 66 minutes, about 68 minutes, about 70 minutes, about 72 minutes, about 74 minutes, about 76 minutes, about 78 minutes, about 80 minutes, about 82 minutes, about 84 minutes, about 86 minutes, about 88 minutes, about 90 minutes, about 92 minutes, about 94 minutes, about 96 minutes, about 98 minutes, about 100 minutes, about 102 minutes, about 104 minutes, about 106 minutes, about 108 minutes, about 110 minutes, about 112 minutes, about 114 minutes, about 116 minutes, about 118 minutes, about 120 minutes, or any range including and/or in between any two of the preceding values. Additionally or alternatively, in some embodiments, the specific temperature and time may be determined based upon factors which may include accuracy of the oven temperature, the material on which the placental tissue is fixed within the oven, the volume of tissue being dehydrated, and the like. Additionally or alternatively, in some embodiments, the placental tissue may be packaged for distribution. Additionally or alternatively, in some embodiments, when ready for use in forming the dressing 100, the placental tissue may be removed from the packaging.

In any embodiment disclosed herein, the method for preparing the dressing may comprise forming a slurry comprising collagen and ORC. Additionally or alternatively, in some embodiments the slurry may further comprise additives, for example, a metal such as silver. Additionally or alternatively, in some embodiments, at least a portion of the metal may be present in a complex with another component, for example, an ORC-silver complex.

In any embodiment disclosed herein, the method for preparing the dressing may comprise applying the slurry to one or more surfaces of the placental tissue and drying the slurry, for example, to form a sponge or a film. Additionally or alternatively, in some embodiments, the slurry may be applied to only one surface of the placental tissue, two surfaces of the placental tissue, or the placental tissue may be disposed within the slurry, depending upon the desired configuration of the dressing.

In any embodiment herein, drying may comprise freeze-drying the slurry, solvent-drying the slurry, or otherwise dehydrating the slurry. Additionally or alternatively, in some embodiments, freeze-drying may comprise freezing the slurry, followed by evaporating the solvent from the frozen slurry under reduced pressure. A method of freeze-drying may be similar to the method described for drying a collagen-based sponge in U.S. Pat. No. 2,157,224, the entire content of which is incorporated herein by reference. Additionally or alternatively, in some embodiments, the freeze-drying may be performed in one or more stages. Solvent-drying may comprise freezing the slurry, followed by immersing the slurry in a series of baths of a hygroscopic organic solvent, such as anhydrous isopropanol, to extract the water from the frozen slurry, followed by removing the organic solvent by evaporation. Methods of solvent drying are described, for example, in U.S. Pat. No. 3,157,524, the entire content of which is incorporated herein by reference. Additionally or alternatively, in some embodiments, the method may further comprise placing the slurry in a dehydration oven, for example, which may evaporate water and/or solvent using suitably higher temperatures with or without circulation of air through a chamber containing a desiccant or the like.

In any embodiment disclosed herein, the method may further comprise treating the slurry, or the dried dressing, with a cross-linking agent such as epichlorhydrin, carbodiimide, hexamethylene diisocyanate (HMDI) orglutaraldehyde. Additionally or alternatively, in some embodiments, cross-linking may be carried out dehydrothermally Additionally or alternatively, in some embodiments, the particular method of cross-linking may be selected based upon a desired final product. For example, HMDI may cross-link the primary amino groups of collagen, whereas carbodiimide may cross-link carbohydrate of the ORC to primary amino groups of the collagen.

Negative-Pressure Therapy

In any embodiment disclosed herein, the dressing 100 may be employed for treatment of a tissue site with reduced pressure.

Clinical studies and practice have shown that reducing pressure in proximity to a tissue site can augment and accelerate growth of new tissue at the tissue site. The applications of this phenomenon are numerous, but it has proven particularly advantageous for treating wounds. Treatment of tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits may increase development of granulation tissue and reduce healing times.

“Negative pressure” generally refers to a pressure less than a local ambient pressure, such as ambient pressure in a local environment external to a sealed therapeutic environment. In many cases, local ambient pressure may also be atmospheric pressure near a tissue site. Alternatively, the pressure may be less than a hydrostatic pressure associated with tissue at a tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure applied to a tissue site may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa). In any embodiment disclosed herein, the negative pressure applied to a wound may be about −5 mm Hg to about −500 mm Hg. Additionally or alternatively, in some embodiments, the negative pressure applied to a wound may be about −5 mm Hg, about −6 mm Hg, about −7 mm Hg, about −8 mm Hg, about −9 mm Hg, about −10 mm Hg, about −11 mm Hg, about −12 mm Hg, about −13 mm Hg, about −14 mm Hg, about −15 mm Hg, about −16 mm Hg, about −17 mm Hg, about −18 mm Hg, about −19 mm Hg, about −20 mm Hg, about −22 mm Hg, about −24 mm Hg, about −26 mm Hg, about −28 mm Hg, about −30 mm Hg, about −32 mm Hg, about −34 mm Hg, about −36 mm Hg, about −38 mm Hg, about −40 mm Hg, about −42 mm Hg, about −44 mm Hg, about −46 mm Hg, about −48 mm Hg, about −50 mm Hg, about −52 mm Hg, about −54 mm Hg, about −56 mm Hg, about −58 mm Hg, about −60 mm Hg, about −62 mm Hg, about −64 mm Hg, about −66 mm Hg, about −68 mm Hg, about −70 mm Hg, about −72 mm Hg, about −74 mm Hg, about −76 mm Hg, about −78 mm Hg, about −80 mm Hg, about −82 mm Hg, about −84 mm Hg, about −86 mm Hg, about −88 mm Hg, about −90 mm Hg, about −92 mm Hg, about −94 mm Hg, about −96 mm Hg, about −98 mm Hg, about −100 mm Hg, about −110 mm Hg, about −120 mm Hg, about −130 mm Hg, about −140 mm Hg, about −150 mm Hg, about −160 mm Hg, about −170 mm Hg, about −180 mm Hg, about −190 mm Hg, about −200 mm Hg, about −220 mm Hg, about −240 mm Hg, about −260 mm Hg, about −280 mm Hg, about −300 mm Hg, about −320 mm Hg, about −340 mm Hg, about −360 mm Hg, about −380 mm Hg, about −400 mm Hg, about −420 mm Hg, about −440 mm Hg, about −460 mm Hg, about −480 mm Hg, about −500 mm Hg, or any range including and/or in between any two of these values.

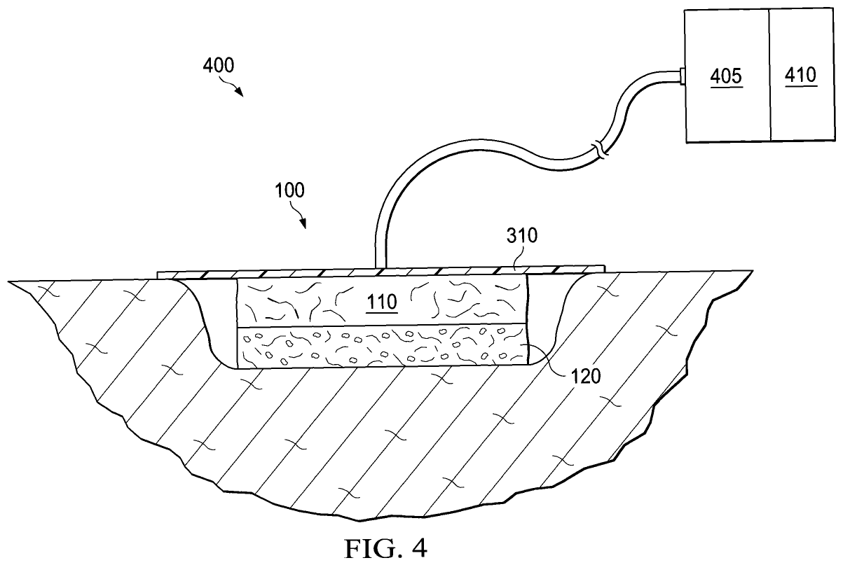

FIG. 4 is a simplified functional block diagram of an example embodiment of a therapy system 400 with an embodiment of the dressing 100 that can provide negative-pressure therapy to a tissue site. Additionally or alternatively, in some embodiments, the therapy system 400 may include a source or supply of negative pressure, such as a negative-pressure source 405, and a regulator or controller, such as a controller 410. Additionally or alternatively, in some embodiments, the therapy system 400 may include sensors to measure operating parameters and provide feedback signals to the controller 410 indicative of the operating parameters. Additionally or alternatively, in some embodiments, the therapy system 400 may include a pressure sensor, an electric sensor, or both, coupled to the controller 410.

In any embodiment disclosed herein, the therapy system may also include various distribution components, such as the dressing 100 and a fluid container. In general, a distribution component may refer to any complementary or ancillary component configured to be fluidly coupled to a negative-pressure supply in a fluid path between a negative-pressure supply and a tissue site. For example, in the embodiment of FIG. 4, the dressing 100 is fluidly coupled to the negative-pressure source 405 such that negative pressure may be applied to a tissue site via the dressing 100.

In the example of FIG. 4, the dressing 100 may be generally configured to distribute negative pressure, to collect fluid, or both. Additionally or alternatively, in some embodiments, one or more layers of the dressing 100 may comprise or be configured as a manifold. A “manifold” in this context generally includes any composition or structure providing a plurality of pathways configured to collect or distribute fluid across a tissue site under pressure. Additionally or alternatively, in some embodiments, a manifold may be configured to receive negative pressure from a negative-pressure source and to distribute negative pressure through multiple apertures (e.g., pores) across a tissue site, which may have the effect of collecting fluid and drawing the fluid toward the negative-pressure source. In the embodiment of FIG. 4, the dressing 100 is configured to receive negative pressure from the negative-pressure source 405 and to distribute the negative pressure through the dressing 100. Additionally or alternatively, in some embodiments, the fluid path may be reversed or a secondary fluid path may be provided to facilitate movement of fluid across a tissue site.

In any embodiment disclosed herein, the fluid pathways of a manifold may be interconnected to improve distribution or collection of fluids. Additionally or alternatively, in some embodiments, a manifold may be a porous foam material having interconnected cells or pores. For example, reticulated and other open-cell foam generally includes pores, edges, and/or walls that may form interconnected fluid pathways.

The average pore size of foam may vary according to needs of a prescribed therapy. In any embodiment disclosed herein, one or more layers of the dressing 100 may comprise foam having pore sizes in a range of about 400 microns to about 600 microns. Additionally or alternatively, in some embodiments, one or more layers of the dressing 100 may comprise foam having pore sizes in a range of about 400 microns, about 420 microns, about 440 microns, about 460 microns, about 480 microns, about 500 microns, about 520 microns, about 540 microns, about 560 microns, about 580 microns, about 600 microns, or any range including and/or in between any two of these values. The tensile strength may also vary according to needs of a prescribed therapy. Additionally or alternatively, in some embodiments, one or more layers of the dressing 100 may comprise reticulated polyurethane foam such as found in GRANUFOAM™ dressing or V.A.C. VERAFLO™ dressing, both available from KCI of San Antonio, Tex.

The fluid mechanics associated with using a negative-pressure source to reduce pressure in another component or location, such as within a sealed therapeutic environment, can be mathematically complex. However, the basic principles of fluid mechanics applicable to negative-pressure therapy are generally well-known to those skilled in the art, and the process of reducing pressure may be described illustratively herein as “delivering,” “distributing,” or “generating” negative pressure, for example.

In general, a fluid, such as wound fluid (for example, wound exudates and other fluids), flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. This orientation is generally presumed for purposes of describing various features and components herein. However, the fluid path may also be reversed in some applications (such as by substituting a positive-pressure source for a negative-pressure source) and this descriptive convention should not be construed as a limiting convention.

In any embodiment disclosed herein, a negative-pressure supply, such as the negative-pressure source 405, may be a reservoir of air at a negative pressure, or may be a manual or electrically-powered device that can reduce the pressure in a sealed volume, such as a vacuum pump, a suction pump, a wall suction port available at many healthcare facilities, or a micro-pump, for example. Additionally or alternatively, in some embodiments, a negative-pressure supply may be housed within or used in conjunction with other components, such as sensors, processing units, alarm indicators, memory, databases, software, display devices, or user interfaces that further facilitate therapy. Additionally or alternatively, in some embodiments, the negative-pressure source 405 may be combined with a controller and other components into a therapy unit. Additionally or alternatively, in some embodiments, a negative-pressure supply may also have one or more supply ports configured to facilitate coupling and de-coupling of the negative-pressure supply to one or more distribution components.

In any embodiment disclosed herein, components may be fluidly coupled to each other to provide a path for transferring fluids (i.e., liquid and/or gas) between the components. For example, components may be fluidly coupled through a fluid conductor, such as a tube. As used herein, the term “fluid conductor” is intended to broadly include a tube, pipe, hose, conduit, or other structure with one or more lumina or pathways adapted to convey a fluid between two ends thereof. Additionally or alternatively, in some embodiments, a fluid conductor may be an elongated, cylindrical structure with some flexibility, but the geometry and rigidity may vary. Additionally or alternatively in some embodiments, the negative-pressure source 405 may be operatively coupled to the dressing 100 via a dressing interface. For example, in the embodiment of FIG. 4, the dressing 100 may be coupled to the negative-pressure source 405 via a dressing interface such that the dressing 100 receives negative pressure from the negative-pressure source 405.

Methods of Use

Also disclosed herein are methods of treating a tissue site, for example, in the context of various therapies.

In any embodiment disclosed herein, a therapy method may comprise applying the dressing 100 to a tissue site. Additionally or alternatively, in some embodiments, in operation, the dressing 100 may be positioned within, over, on, or otherwise proximate to a tissue site. Additionally or alternatively, in some embodiments, the dressing 100 may include a cover, such as the cover 310, that may be sealed to an attachment surface near a tissue site. For example, the cover 310 may be sealed to undamaged epidermis peripheral to a tissue site. Additionally or alternatively, in some embodiments, the components of the dressing 100 may be positioned sequentially. Additionally or alternatively, in some embodiments, the dressing 100 may be preassembled, for example, such that the cover 310 is positioned with respect to other components of the dressing 100 prior to placement proximate a tissue site. Thus, the cover 310 can seal the any other layers of the dressing 100 in a therapeutic environment proximate to a tissue site, substantially isolated from the external environment. Additionally or alternatively, in some embodiments, the cover may be configured to be positioned over the dehydrated placental component 110.

Additionally or alternatively, in some embodiments, a therapy method may further comprise fluidly coupling a negative-pressure source to a dressing, such as the dressing 100, and operating the negative-pressure source to generate a negative pressure proximate to a tissue site. For example, the negative-pressure source 405 may be coupled to the dressing 100 such that the negative-pressure source 405 may be used to reduce the pressure beneath the cover 310. Additionally or alternatively, in some embodiments, negative pressure applied across a tissue site, for example, via the dressing 100, may be effective to induce macrostrain and microstrain at the tissue site, as well as remove exudate and other fluids from the tissue site. Additionally or alternatively, in some embodiments, exudate and other fluid may be stored in one or more layers of the dressing 100. Additionally or alternatively, in some embodiments, exudate and other fluid can transferred to an external container.

In another aspect, the present disclosure provides a method for treating a wound in a subject in need thereof, wherein the method comprises administering to the wound a dressing of any embodiment disclosed herein. Additionally or alternatively, in some embodiments of the methods disclosed herein, the wound may be an acute wound or a chronic wound. Additionally or alternatively, in some embodiments of the methods disclosed herein, the wound is an acute wound selected from the group consisting of burns, skin grafts, and dehisced surgical wounds. Additionally or alternatively, in some embodiments of the methods disclosed herein, the wound is a chronic wound selected from the group consisting of infectious wounds, venous ulcers, arterial ulcers, decubitis ulcers and diabetic ulcers.

Any method known to those in the art for administering a dressing to an acute wound or a chronic wound disclosed herein may be employed. Suitable methods include in vitro or in vivo methods. In vivo methods typically include the administration of one or more dressings to a subject in need thereof, suitably a human. In some embodiments of the methods disclosed herein, the dressing is applied directly to the wound. When used in vivo for therapy, the one or more dressings described herein are administered to the subject in effective amounts (i.e., amounts that have desired therapeutic effect). The dose and dosage regimen will depend upon the state of the wound of the subject, and the characteristics of the particular wound dressing used.

The effective amount may be determined during pre-clinical trials and clinical trials by methods familiar to physicians and clinicians. An effective amount of one or more dressings useful in the methods may be administered to a subject in need thereof by any number of well-known methods for administering dressings.

In some embodiments of the methods disclosed herein, the dressings are administered daily for 1 hour or more, for 2 hours or more, for 3 hours or more, for 4 hours or more, for 5 hours or more, for 6 hours or more, for 12 hours or more. Additionally or alternatively, in some embodiments of the methods disclosed herein, the dressings are administered one, two, three, four, or five times per day. Additionally or alternatively, in some embodiments of the methods disclosed herein, the dressings are administered daily for one, two, three, four or five weeks. Additionally or alternatively, in some embodiments of the methods disclosed herein, the dressings are administered daily for less than 6 weeks. Additionally or alternatively, in some embodiments of the methods disclosed herein, the dressings are administered daily for 6 weeks or more. Additionally or alternatively, in some embodiments of the methods disclosed herein, the dressings are administered daily for 12 weeks or more. Additionally or alternatively, in some embodiments of the methods disclosed herein, the dressings are administered every day, every other day, every third day, every fourth day, every fifth day, or every sixth day. Additionally or alternatively, in some embodiments of the methods disclosed herein, the dressings are administered weekly, bi-weekly, tri-weekly, or monthly. Additionally or alternatively, in some embodiments of the methods disclosed herein, the dressings are administered for a period of one, two, three, four, or five weeks. Additionally or alternatively, in some embodiments of the methods disclosed herein, the dressings are administered for six weeks or more. Additionally or alternatively, in some embodiments of the methods disclosed herein, the dressings are administered for twelve weeks or more. Additionally or alternatively, in some embodiments of the methods disclosed herein, the dressings are administered for a period of less than one year. Additionally or alternatively, in some embodiments of the methods disclosed herein, the dressings are administered for a period of more than one year.

In some embodiments of the methods disclosed herein, the dressings can be changed for a chronic wound as appropriate. Additionally or alternatively, in some embodiments of the methods disclosed herein, the wound is a chronic wound selected from the group consisting of infectious wounds, venous ulcers, arterial ulcers, decubitis ulcers and diabetic ulcers.

Method of Making the Dressings of the Present Technology

Also disclosed herein are methods for making the wound dressings of the present technology. In one aspect, the present disclosure provides a method of making a dressing, the method comprising providing dehydrated placental tissue, a collagen, and an oxidized regenerated cellulose (ORC); optionally wherein the dehydrated placental tissue, the collagen, and the ORC are present in a single layer.

In another aspect, the present disclosure provides a method of making a dressing, the method comprising providing a first layer and a second layer; wherein the first layer comprises an effective amount of dehydrated placental tissue, wherein the second layer comprises a collagen and an oxidized regenerated cellulose (ORC), wherein the second layer comprises about 50% to about 60% collagen by weight, and wherein the second layer comprise about 40% to about 50% ORC by weight.

In any embodiment disclosed herein, the dressing is in the form of a sponge. Additionally or alternatively, in some embodiments, the dressing is in the form of a film.

Additionally or alternatively, in some embodiments, the dressing is in the form of a sponge. Additionally or alternatively, in some embodiments, the dressing is in the form of a film.

In any embodiment disclosed herein, the method may further comprise

For example, in some embodiments, a dressing including a DHAM and a collagen/ORC sponge may be prepared. An initial slurry may be generated through the swelling of a collagen in 0.05M acetic acid solution. Once the collagen is sufficiently swelled, powdered ORC is blended into the collagen slurry. The resulting slurry mixture has a solids content of about 1%, with collagen and ORC present at a ratio of 55%:45%, respectively. The slurry mixture is then decanted into a suitable tray at which point a sheet of DHAM is applied directly to the surface of the slurry mixture. The slurry is then immediately transferred to a freezer at a temperature of about −70° C. Once frozen, the block is freeze-dried, producing a single dressing with two distinct layers: a DHAM layer and a collagen/ORC sponge layer.

For example, in some embodiments, a dressing including a DHAM and a collagen/ORC film may be prepared. An initial slurry may be generated through the swelling of a collagen in 0.05M acetic acid solution. Once the collagen is sufficiently swelled, powdered ORC is blended into the collagen slurry. The resulting slurry mixture has a solids content of about 1%, with collagen and ORC present at a ratio of 55%:45%, respectively. Next, glycerol (300 μl glycerol per 100 ml collagen/ORC slurry) was added as a plasticizer. The slurry mixture is then decanted into a suitable container and degassed in a vacuum. Once degassed, the slurry mixture is poured into a suitable tray (˜31 g of slurry per 10×10 cm) and a sheet of DHAM is applied directly to the surface of the slurry. This composite material is then dehydrated for about 24 hours at 37° C. The resulting dehydration produces a single dressing with two distinct layers: a DHAM layer and a collagen/ORC film layer.

Kits

The present disclosure provides kits that include a dressing of any embodiment described herein and instructions for use. The kit may optionally include instructions for generating a dressing of any embodiment described herein. The kits may further comprise additional canisters, drapes, medical-grade adhesive, or spare tubing. The kit may optionally comprise components such as antiseptic wipes, ointment, adhesive tape, tweezers, or scissors.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds, compositions, or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third, and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

The advantages and features associated with the compositions, dressings, and various other embodiments described herein can be further demonstrated by the following, non-limiting examples.

### EXAMPLES

### Example 1—DHAM and Collagen/ORC Sponge

In a first example, a dressing including a DHAM and a collagen/ORC sponge was prepared. In the first example, an initial slurry was generated through the swelling of collagen in 0.05M acetic acid solution. Once the collagen was sufficiently swelled, powdered ORC was blended into the collagen slurry. The resulting slurry mixture had a solids content of about 1%, with collagen and ORC present at a ratio of 55%:45%, respectively. The slurry mixture was then decanted into a suitable tray at which point a sheet of DHAM was applied directly to the surface of the slurry mixture. This was then immediately transferred to a −70° C. freezer. Once frozen, the block was freeze-dried, producing a single dressing with two distinct layers: a DHAM layer and a collagen/ORC sponge layer.

### Example 2—DHAM and Collagen/ORC Film

In a second example, a dressing including a DHAM and a collagen/ORC film was prepared. In the second example, an initial slurry material was generated through the swelling of collagen in 0.05M acetic acid solution. Once the collagen was sufficiently swelled, powdered ORC was blended into the collagen slurry. The resulting slurry mixture had a solids content of about 1%, with collagen and ORC present at a ratio of 55%:45%, respectively. Further to the collagen and ORC, glycerol (300 μl glycerol per 100 ml collagen/ORC slurry) was added as a plasticizer. The resulting slurry mixture was then decanted into a suitable container and degassed in a vacuum. Once degassed, the slurry mixture was poured into a suitable tray (˜31 g of slurry per 10×10 cm) and a sheet of DHAM applied directly to the surface of the slurry. This was dehydrated for about 24 hours at 37° C. The resulting dehydration produced a single dressing with two distinct layers: a DHAM layer and a collagen/ORC film layer.

### Example 3—Improved Collagen Synthesis Upon Application of the Dressings of the Present Technology

A collagen synthesis assay with dermal fibroblasts is performed. This is a standard assay which shows the amount of collagen synthesized by fibroblasts after stimulation with the active agents in the dressings of the present technology. Briefly, 8.4×10⁴ human fibroblasts (per well) are plated into 24-well plates, and then incubated at 37° C., 5% CO₂, in 10% FBS-DMEM. Once the cells are confluent (within 24 hours of plating), the 10% FBS-DMEM is removed, and the cells are washed 3× with serum-free DMEM (SF-DMEM), before the test dressing samples of the present technology or a collagen/ORC alone dressing is added to the cells. Cells are then incubated for 72 hours after which time the media is collected and analyzed for the levels of the C-terminal propeptide of Type-1 Collagen (CICP) present in the cell culture media. The level of CICP in the media, which is released by the fibroblasts: as a by-product of collagen synthesis, is proportional to the level of collagen synthesis and so its level was used to determine the level of collagen synthesis. It is anticipated that the dressings of the present technology will exhibit increased collagen synthesis and improved wound healing of chronic or acute wounds compared to a standard collagen/ORC dressing.

Further, the collagen synthesis assay with dermal fibroblasts: is performed with the dressings of the present technology as well as a conventional wound dressing (e.g., Grafix®, Osiris Therapeutics, Inc Columbia, Md.). Following the incubation of dermal fibroblast cells with SF-DMEM extracts of the dressing samples, it is anticipated that the dressings of the present technology exhibit increased collagen synthesis and improved wound healing of chronic or acute wounds compared to conventional placental wound dressings.

Without wishing to be bound by theory, it is believed that the dressings of the present technology may advantageously reduce wound bioburden, promote cellular and/or vascular invasion into the wound, provide a scaffold for wound healing, and provide extracellular matrix proteins and/or signaling molecules, or combinations thereof. Accordingly, the dressings of the present technology are useful in methods for treating a wound in a subject in need thereof, wherein the method comprises administering to the wound a dressing of any embodiment disclosed herein.

### Example 4—Decreased Protease Activity Upon Application of the Dressings of the Present Technology

A fluorometric assay is utilized to determine residual protease activities after incubation with the test wound dressing samples. Briefly, samples of the wound dressings of the present technology (with or without silver), a standard collagen/ORC dressing, or a conventional placental dressing are incubated either in a solution of simulated wound fluid (SWF) containing human neutrophil elastase (HNE, 273 mU/mL) for 24 hours at 37° C., or in a solution of SWF containing matrix metalloproteinase-9 (MMP-9, 1 μg/mL) for 2 hours at 37° C.

Following incubation, samples of the protease containing SWF are then evaluated by fluorometric assay to quantify residual protease activities. It is anticipated that the dressings of the present technology exhibit reduced protease levels and improved wound healing of chronic or acute wounds compared to standard collagen/ORC dressings and/or conventional placental wound dressings.

Without wishing to be bound by theory, it is believed that the dressings of the present technology may advantageously reduce protease acclivity, reduce wound bioburden, promote cellular and/or vascular invasion into the wound, provide a scaffold for wound healing, and provide extracellular matrix proteins and/or signaling molecules, or combinations thereof. Accordingly, the dressings of the present technology are useful in methods for treating a wound in a subject in need thereof, wherein the method comprises administering to the wound a dressing of any embodiment disclosed herein.

1. A dressing comprising:
dehydrated placental tissue;
a collagen; and
an oxidized regenerated cellulose (ORC), and
optionally wherein the dehydrated placental tissue, the collagen, and the ORC are present in a single layer.

2. The dressing of claim 1, wherein the dehydrated placental tissue is present in a first layer and the collagen and the ORC are combined into a second layer;
optionally wherein a cover is configured to be positioned over the first layer comprising the dehydrated placental tissue; and
optionally wherein the dehydrated placental tissue comprises amniotic membrane tissue, chorion tissue, or a combination thereof.

3. (canceled)

4. The dressing of claim 2, wherein the second layer comprises about 50% to about 60% collagen by weight, and/or about 40% to about 50% ORC by weight,
optionally wherein the second layer is in a freeze-dried form, in the form of a film, or in the form of a sponge; and
optionally wherein the second layer is configured as a wound contact layer.

5. (canceled)

6. (canceled)

7. (canceled)

8. (canceled)

9. (canceled)

10. The dressing of claim 2, wherein the second layer comprises an antimicrobial agent,
optionally wherein the antimicrobial agent is one or more of tetracycline, penicillins, terramycins, erythromycin, bacitracin, neomycin, polymycin B, mupirocin, clindamycin, colloidal silver, silver sulfadiazine, chlorhexidine, povidone iodine, triclosan, sucralfate, quaternary ammonium salts, pharmaceutically acceptable silver salts, or any combination thereof; and
optionally wherein the second layer comprises silver and wherein at least a portion of the silver may be present in a complex with the ORC (silver-ORC).

11. (canceled)

12. (canceled)

13. The dressing of claim 2, wherein the second layer comprises an antioxidant,
optionally wherein the antioxidant comprises one or more of anthocyanins, astaxanthin, bilirubin, canthaxanthin, capsaicin, citric acid, curcumin, coenzyme Q10, eugenol, flavanols, flavonolignans, flavanones, flavones, flavonols, iodide, isoflavone phytoestrogens, lutein, lycopene, manganese, melatonin, N-acetylcysteine, oxalic acid, phenolic acids, phytic acid, R-α-lipoic acid, stilbenoids, tocopherol, tocotrienol, vitamin A, vitamin C, vitamin E, xanthones, zeaxanthin, α-carotene, β-carotene, or any combination thereof.

14. (canceled)

15. (canceled)

16. A method of wound therapy comprising applying the dressing of claim 2 to a tissue site,
optionally wherein the second layer is configured as a wound contact layer; and
optionally wherein the therapy is negative pressure wound therapy.

17. (canceled)

18. (canceled)

19. The method of claim 16, further comprising sealing the dressing to tissue surrounding the tissue site to form a sealed space, optionally wherein sealing the dressing to tissue surrounding the tissue site comprises sealing a cover to the tissue surrounding the tissue site.

20. (canceled)

21. The method of claim 19, further comprising:
fluidly coupling a negative-pressure source to the sealed space; and
operating the negative-pressure source to generate a negative pressure in the sealed space.

22. A dressing comprising:
a first layer and a second layer;
wherein the first layer comprises an effective amount of dehydrated placental tissue,
wherein the second layer comprises a collagen and an oxidized regenerated cellulose (ORC),
wherein the second layer comprises about 50% to about 60% collagen by weight, and
wherein the second layer comprise about 40% to about 50% ORC by weight,
optionally wherein the dehydrated placental tissue comprises amniotic membrane tissue, chorion tissue, or a combination thereof, and
optionally wherein a cover is configured to be positioned over the first layer comprising the dehydrated placental tissue.

23. (canceled)

24. The dressing of claim 22, wherein the second layer is in
a freeze-dried form, in the form of a film, or in the form of a sponge; and
optionally wherein the second layer is configured as a wound contact layer.

25. (canceled)

26. (canceled)

27. (canceled)

28. The dressing of claim 22, wherein the second layer comprises an antimicrobial agent,
optionally wherein the antimicrobial agent is one or more of tetracycline, penicillins, terramycins, erythromycin, bacitracin, neomycin, polymycin B, mupirocin, clindamycin, colloidal silver, silver sulfadiazine, chlorhexidine, povidone iodine, triclosan, sucralfate, quaternary ammonium salts, pharmaceutically acceptable silver salts, or any combination thereof; and
optionally wherein the second layer comprises silver and wherein at least a portion of the silver may be present in a complex with the ORC (silver-ORC).

29. (canceled)

30. (canceled)

31. The dressing of claim 22, wherein the second layer comprises an antioxidant, optionally wherein the antioxidant comprises one or more of anthocyanins, astaxanthin, bilirubin, canthaxanthin, capsaicin, citric acid, curcumin, coenzyme Q10, eugenol, flavanols, flavonolignans, flavanones, flavones, flavonols, iodide, isoflavone phytoestrogens, lutein, lycopene, manganese, melatonin, N-acetylcysteine, oxalic acid, phenolic acids, phytic acid, R-α-lipoic acid, stilbenoids, tocopherol, tocotrienol, vitamin A, vitamin C, vitamin E, xanthones, zeaxanthin, α-carotene, β-carotene, or any combination thereof.

32. (canceled)

33. (canceled)

34. A method of wound therapy comprising applying the dressing of claim 22 to a tissue site,
optionally wherein the second layer is configured as a wound contact layer, and
optionally wherein the therapy is negative pressure wound therapy.

35. (canceled)

36. (canceled)

37. The method of claim 34, further comprising sealing the dressing to tissue surrounding the tissue site to form a sealed space,
optionally wherein sealing the dressing to tissue surrounding the tissue site comprises sealing a cover to the tissue surrounding the tissue site.

38. (canceled)

39. The method of claim 37, further comprising:
fluidly coupling a negative-pressure source to the sealed space; and
operating the negative-pressure source to generate a negative pressure in the sealed space.

40. A method for treating a wound in a subject in need thereof, the method comprising administering to the wound a dressing of claim 22, optionally wherein the wound dressing is applied directly to the wound and/or optionally wherein the wound is an acute wound or a chronic wound.

41. (canceled)

42. (canceled)

43. A method for making a dressing, comprising:
providing dehydrated placental tissue, a collagen, and an oxidized regenerated cellulose (ORC); optionally wherein the dehydrated placental tissue, the collagen, and the ORC are present in a single layer or
providing a first layer and a second layer,
wherein the first layer comprises an effective amount of dehydrated placental tissue and the second layer comprises a collagen and an oxidized regenerated cellulose (ORC);
optionally wherein the second layer comprises about 50% to about 60% collagen by weight and/or about 40% to about 50% ORC by weight.

44. (canceled)

45. The method of claim 43, wherein the dressing is in the form of a sponge, or in the form of a film.

46. (canceled)

47. The method of claim 43, wherein the second layer further comprises at least one plasticizer,
optionally wherein the at least one plasticizer comprises an acetylated monoglyceride, an alkyl citrate, methyl ricinoleate, glycerol, and a combination of any two or more thereof, and
optionally wherein the alkyl citrate comprises triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate, trimethyl citrate, or a combination of any two or more thereof.

48. (canceled)

49. (canceled)

50. A kit comprising the dressing of claim 1, and instructions for use.
